(19) United States
(12) Patent Application Publication
(10) Pub. No.: US 2007/0116646 A1
(43) Pub. Date: May 24, 2007

(54) Optical imaging of endometriosis

(76) Inventors: Jo Klaveness, Oslo (NO); Edvin Johannesen, Oslo (NO); Helge Tolleshaug, Oslo (NO)

Correspondence Address: GE HEALTHCARE, INC., IP DEPARTMENT, 101 CARNEGIE CENTER, PRINCETON, NJ, 08540-6231, US

(21) Appl. No.: 10/573,604

(22) PCT Filed: Sep. 28, 2004
(86) PCT No.: PCT/NO04/00286

§ 371 Date: Mar. 28, 2006

(30) Foreign Application Priority Data
Sep. 29, 2003 (NO) 20034351

(51) Int. Cl. A61K 49/00; C07K 16/46

(52) U.S. Cl. 424/9.6; 530/409; 424/178.1; 530/391.1

(57) ABSTRACT

The invention provides contrast agents for optical imaging of endometriosis in patients. The contrast agent may be used in diagnosis of endometriosis, for follow-up of progress in disease development, and for follow-up of treatment of endometriosis. Further, the invention provides methods for optical imaging of endometriosis. The contrast agent has affinity for an abnormally expressed target associated with endometriosis. By abnormally expressed, is meant that the target is either overexpressed or downregulated. Endometriotic tissue containing a downregulated target may be identified by a low amount of bound contrast agent compared to normal tissue. In this situation, the amount of contrast agent should be less than 50% of that in normal tissue, preferably less than 10%.

The following definitions will be used throughout the document:
Endometriotic tissue: The aberrant occurrence of tissue that more or less perfectly resembles the endometrium in various locations in the pelvic cavity.
Abnormally expressed target: A target that is either overexpressed or downregulated in endometriotic tissue.
Overexpressed target: A receptor, an enzyme or another molecule or chemical entity that is present in a higher amount (upregulated) in endometriotic tissue than in normal tissue.
Downregulated target: A receptor, an enzyme or another molecule or chemical entity that is present in a lower amount in endometriotic tissue than in normal tissue.

### DETAILED DESCRIPTION OF THE INVENTION

A first aspect of the present invention is an optical imaging contrast agent for imaging of endometriosis. By the term optical imaging contrast agent, or just contrast agent, we mean a molecular moiety used for enhancement of image contrast in vivo comprising at least one moiety that interacts with light in the ultraviolet, visible or near infrared part of the electromagnetic spectrum.

The contrast agent has affinity for an abnormally expressed target associated with endometriosis. By abnormally expressed, is meant that the target is either overexpressed or downregulated.

Endometriotic tissue containing a downregulated target may be identified by a low amount of bound contrast agent compared to normal tissue. In this situation, the amount of contrast agent should be less than 50% of that in normal tissue, preferably less than 10%.

Preferably, the contrast agent, according to the present invention, has affinify for an overexpressed target associated with endometriosis. Preferred targets are those targets that are more than 50% abundant in endometriotic tissue than in surrounding tissue. More preferred targets are those targets that are more than two times abundant in endometriotic tissue than in surrounding tissue. The most preferred targets are those targets that are more than 5 times abundant in endometriotic tissue than in surrounding tissue.

Relevant groups of targets are receptors, enzymes, nucleic acids, proteins, lipids, and other macromolecules such as for example lipoproteins and glycoproteins. The targets may be located in the vascular system, in the extracellular space, associated with cell membranes or located intracellularly.

The following biological targets are preferred targets for contrast agents for optical imaging of endometriosis:
Overexpressed Targets:
Angiogenesis Targets:

Vascular endothelial growth factor, integrins (β₁-integrins, integrin alpha, beta₃) and matrix metalloproteases.

Mainly upregulated during the secretory phase of the menstrual cycle: matrix metalloproteinase 7
Receptors:

Estrogen receptors, progesterone receptors, interleukin-1 receptor.

Mainly upregulated during the secretory phase of the menstrual cycle: galectin 6-binding protein.
Adhesion Molecules:

ICAM-1, CD44; cadherins such as E-cadherin, N-cadherin, P-cadherin and cadherin 11.
Extracellular Matrix Proteins:

Tenascin, osteopontin, fibulin 1, proteoglycan 4.
Enzymes:

Endothelial nitric oxide synthase, cathepsin H, cathepsin S, superoxide dismutase, aromatase, protein kinases, especially extracellular signal regulated kinase (ERK), HMG CoA reductase, Tyrosine kinases, reductase, protein farnesyltransferase, 17β-hydroxysteroid dehydrogenase, cyclooxygenase-2, Xanthine oxidase, membrane-associated neutral endopeptidase (CD10), catalase and MMPs.

Mainly upregulated during the proliferative phase of the menstrual cycle: ribonuclease, phytanoyl-CoA hydroxylase, pyrroline 5-carboxyjate reductase.

Mainly upregulated during the secretory phase of the menstrual cycle: palmitoyl-protein thioesterase 1, ubiquitin-conjugating enzyme E2N, malate dehydrogenase 1, aldehyde dehydrogenase 1, sterol-C5-desaturase, IGF-binding serine protease, α-L-fucosidase, glycogenin.
Oncogenes and Neoplasia-related Proteins:

c-myc, c-erb-B2, nm23, hepatocellular carcinoma-associated antigen 112, acute lymphoblastic leukemia antigen (CD10) and p53.

Mainly upregulated during the proliferative phase of the menstrual cycle: Src-like adaptor protein, Ras suppressor protein 1.

Mainly upregulated during the secretory phase of the menstrual cycle: neuroblastoma suppressor 1.
Cytokines and Similar Signal Proteins:

Interleukin-6, monocyte chemotactic protein-1, transforming growth factor, IgE-dependent histamine-releasing factor

Mainly upregulated during the proliferative phase of the menstrual cycle: Small inducible cytokine A4.
Proteins of the Immune System:

C3 complement, complement component 1S subcomponent, major histocompatibility antigens, class II, particularly DP α1, DQ α1, DR α, DQ β1, DR β and major histocompatibility complex class 1C, lg-lambda light chain, Ig H chain G-E-A region gamma-2 constant region.

Mainly upregulated during the secretory phase of the menstrual cycle: Complement component 3, properdin, complement component 1s, complement component 1r, complement component 2, Major histocompatibility antigens (class I F).
Cytoskeletal Proteins:

β-actin, α₂ actin, vimentin.

Mainly upregulated during the proliferative phase of the menstrual cycle: Actin-related protein 2/3 complex (subunit 1A), myosin regulatory light chain 2 (smooth muscle isoform), tropomyosin 1, β-actin.

Transport Proteins:

Folate binding proteins, haptoglobin.

Mainly upregulated during the proliferative phase of the menstrual cycle: lactotransferrin, cellular retinol-binding protein, lysosomal H⁺-transporting ATP-ase, potassium voltage-gated channel (shaker-related subfamily, member 5).

Mainly upregulated during the secretory phase of the menstrual cycle: heme-binding protein.
Ribosomal Proteins:

40S ribosomai protein S23.

Mainly upregulated during the proliferative phase of the menstrual cycle: ribosomal protein L11, ribosomal protein S11, ubiquitin A-52 residue ribosomal protein fusion product 1.

Mainly upregulated during the secretory phase of the menstrual cycle: ribosomal protein 823.
Others:

CD14 (lipopolysaccharide binding protein), CD43, CD45, endoglin, Endometrial bleeding-associated factor (LEFTY-A), arachidonate 5-lipooxygenase-activating protein.

Mainly upregulated during the proliferative phase of the menstrual cycle: CDC10, S100 calcium-binding protein A13, tax interaction protein 1, GDP dissociation inhibitor 2, chimaerin 2.

Mainly upregulated during the secretory phase of the menstrual cycle: CCMT/enhancer binding protein, epididymai secretory protein, low-density lipoprotein-related protein-associated protein 1, cyclin D2, caveolin 1, cellular repressor of E1A-stimulated genes, G-rich RNA sequence binding factor 1.
Downregulated Targets:
Angiogenesis Targets:

Mainly downregulated during the proliferative phase of the menstrual cycle: cysteine-rich angiogenic inducer.
Receptors:

Arginine vasopressin receptor 1A. Mainly downregulated during the proliferative phase of the menstrual cycle: G protein-coupled receptor RDC1.

Mainly down regulated during the secretory phase of the menstrual cycle: formyl peptide receptor-like 1.
Adhesion Molecules:

Mainly downregulated during the secretory phase of the menstrual cycle: R-cadherin (cadherin 4), vascular endothelial junction-associated molecule.
Extracellular Matrix Proteins:

Mainly downregulated during the proliferative phase: Laminin β3.

Mainly downregulated during the secretory phase of the menstrual cycle: crystallin αB.
Enzymes: Arginase, mitogen-activated protein kinase kinase 3, cytochrome P450 2C18, glycerol kinase, serum/gucocorticoid regulated kinase, long-chain fatty acid coenzyme A ligase 5, RNA helicase.

Mainly downregulated during the proliferative phase of the menstrual cycle: carbonic anhydrase XII, pantetheinase (vanin 1).

Mainly downregulated during the secretory phase of the menstrual cycle: glutathione peroxidase, monoamine oxidase, histone deacetylase, phosphatidylserine decarboxylase, serine/threonine kinase 19, myo-inositol-monophosphatase, glycogen phosphorylase, alkylglycerone phosphate synthase, creatine kinase, phenylalanyl-tRNA synthetase β-subunit.
Oncogenes and Neoplasia-related Proteins:

Retinoblastoma-binding protein 6, RAS-dexamethason-induced 1.

Mainly downregulated during the proliferative phase of the menstrual cycle: N-myc (downstream regulated)

Mainly downregulated during the secretory phase of the menstrual cycle: growth arrest and DNA-damage-inducible protein-α, retinoblastoma-like 1
Cytokines and Similar Signal Proteins:

lymphotoxin-α, neurotensin

Mainly downregulated during the proliferative phase of the menstrual cycle: cytokine subfamily A member 20.

Mainly downregulated during the secretory phase of the menstrual cycle: cytokine subfamily B member 14, colony-stimulating factor 3, interferon-related developmental regulator 1.
Proteins of the Immune System:

Mainly downregulated during the secretory phase of the menstrual cycle: C4-binding protein-α.
Cytoskeletal Proteins:

Mainly downregulated during the secretory phase of the menstrual cycle: Actin γ2, actinin α4, plectin 1.
Transport Proteins:

Mainly downregulated during the secretory phase of the menstrual cycle: solute carrier family 3 member 1.
Ribosomal Proteins:

Mainly downregulated during the secretory phase of the menstrual cycle: mitochondrial ribosomal protein S2.
Others:

Kruppel-like factor 5, GATA-binding protein 2, heat shock protein 105 kDa, non-histone chromosomal proteins I and Y. oviducal glycoprotein 1 (oviductin), DNAJ-like HSP 2, DNAJ (Hsp40) homolog subfamily B member 1, stromal antigen 2, heat shock protein 70, heat shock protein 27.

Mainly downregulated during the proliferative phase of the menstrual cycle: Zinc finger protein 216, adipose differentiation-reilated protein, heterogeneous nuclear ribonucleoprotein A1, stanniocalcin 1, Golgi membrane protein GP73.

Mainly downregulated during the secretory phase of the menstrual cycle: Anaphase-promoting complex subunit 4, H1 histone-family member 0, t-complex-associated-testis-expressed 1-like, regulator of G-protein signalling 9, islet cell autoantigen 1, metal-regulatory transcription factor 1, heat shock protein 70 kDa protein 1A, thyrotropin-releasing hormone; metallothionein 1E.

Among the most preferred targets for contrast agents for optical imaging of endometriosis are angiogenesis targets, adhesion molecules, estrogen receptors, progesterone receptors, Cathepsin H-and Cathepsin S, aromatase, reductase, CD10, endoglin, haptoglobin and cyclin D2.

Generally, any targets that have been identified as possible targets for agents for treatment of endometriosis are potential targets also in optical imaging.

The preferred contrast agents are molecules with relatively low molecular weights. The molecular weight of preferred contrast agents is below 10000 Daltons, more preferably below 7000 Daltons.

The contrast agents are comprised of a vector that has affinity to an abnormally expressed target in endometriotic tissue, and an optical reporter. Thus viewed from one aspect the present invention provides a contrast agent of formula I:
V-L-R (I)
wherein V is one or more vector moieties having affinity for one or more abnormally expressed target in endometriosis, L is a linker moiety or a bond and R is one or more reporter moieties detectable in optical imaging.

The vector has the ability to direct the contrast agent to a region of endometriosis. The vector has affinity for the abnormally expressed target and preferably binds to the target. The reporter is detectable in an optical imaging procedure and the linker must couple vector to reporter, at least until the reporter has been delivered to the region of endometriosis and preferably until the imaging procedure has been completed.

The vector can generally be any type of molecules that have affinity for the abnormally expressed target. The molecules should be physiologically acceptable and should preferably have an acceptable degree of stability. The vectors can for instance be selected from the following group of compounds: peptides, peptoids/peptidomimetics, oligonucleotides, oligosaccharides, lipid-related compounds, like fatty acids, traditional organic drug-like small molecules, synthetic or semi-synthetic, and derivatives and mimetics thereof. When the target is an enzyme the vector may comprise an inhibitor of the enzyme. The vector of the contrast agent preferably has a molecular weight of less than 4500 Daltbns and more preferably less than 2500 Daltons.

Contrast agents having affinity for more than one abnormally expressed target related to the disease is an aspect of the invention. Such contrast agents can comprise two or more different vectors or molecular subunits that target two or more different abnormally expressed targets.

Another possibility according to the present invention is that the contrast agent comprises one vector that is able to bind to more than one abnormally expressed target in endometriosis.

A contrast agent according to the present invention can also comprise more than one vector of same chemical composition that bind to the abnormally expressed biological target.

Some receptors are unique to endothelial cells and surrounding tissues. Examples of such receptors include growth factor receptors such as VEGF and adhesion molecules such as the integrin family of receptors. Peptides comprising the sequence arginine-glycine-aspartic acid (RGD) are known to bind to a range of integrin receptors. Such RGD-type peptides constitute one group of vectors for targets associated with endometriosis.

Below are some examples of vectors having affinity for endometriosis-related abnormally expressed targets.

Vectors for Angiogenesis Targets,

Vector I: Vectors for integrins—RGD-type peptides having affinity for αᵥβ₃ and αᵥβ₅ such as e.g. 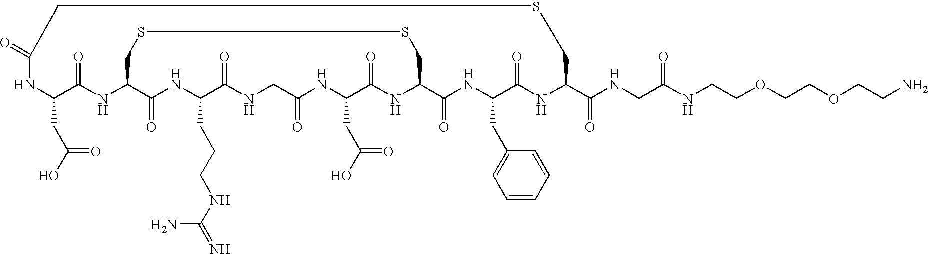
Vector for Progesterone Receptors:

Vector II: Progesterone or a derivative thereof 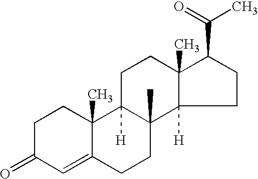
Vectors for Estrogen Receptors:

Vector III: Estrogen, estrogen mimetics or a derivative thereof, e.g. 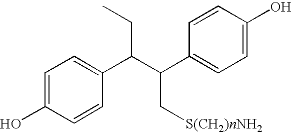
n=2-7

Coupling, takes place via the amine group

Vector for Folate Binding Proteins:

Vector IV: Folate or a derivative thereof

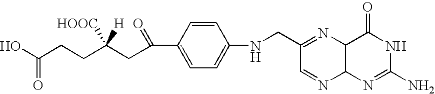

A wide variety of linkers can be used. The linker component of the contrast agent is at its simplest a bond between the vector and the reporter moieties. In this aspect the reporter part of the molecule is directly bound to the molecule sub-unit that binds to the abnormally expressed target. More generally, however, the linker will provide a mono- or multi-molecular skeleton covalently or non-covalently linking one or more vectors to one or more reporters, e.g. a linear, cyclic, branched or reticulate molecular skeleton, or a molecular aggregate, with in-built or pendant groups which bind covalently or non-covalently, e.g. coordinatively, with the vector and reporter moieties. The linker group can be relatively large in order to build into the contrast agent optimal size or optimal shape or simply to improve the binding characteristics for the contrast agent to the abnormally expressed target in endometriotic tissue.

Thus, linking of a reporter unit to a desired vector may be achieved by covalent or non-covalent means, usually involving interaction with one or more functional groups located on the reporter and/or vector. Examples of chemically reactive functional groups which may be employed for this purpose include amino, hydroxyl, sulfhydroxyl, carboxyl and carbonyl groups, as well as carbohydrate groups, vicinal diols, thioethers, 2-aminoalcohols, 2-aminothiols, guanidinyl, imidazolyl and phenolic groups.

The reporter is any moiety capable of detection either directly or indirectly in an optical imaging procedure. The reporter might be a light scatterer (e.g. a coloured or uncoloured particle), a light absorber or a light emitter. More preferably the reporter is a dye such as a chromophore or a fluorescent compound. The dye part of the contrast agent can be any dye that interacts with light in the electromagnetic spectrum with wavelenghts from the ultraviolet light to the near infrared. Preferably, the contrast agent of the invention has fluorescent properties.

Preferred organic chromophoric and fluorophoric reporters include groups having an extensive delocalized electron system, eg. cyanides, merocyanines, indocyanines, phthalocyanines, naphthalocyanines, triphenylmethines, porphyrins, pyrilium dyes, thiapyrilium dyes, squarylium dyes, croconium dyes, azulenium dyes, indoanilines, benzophenoxazinium dyes, benzothiaphenothiazinium: dyes, anthraquinones, napthoquinones, indathrenes, phthaloylacridones, trisphenoquinones, azo dyes, intramolecular and intermolecular charge-transfer dyes and dye complexes, tropones, tetrazines, bis(dithiolene) complexes, bis(benzene-dithiolate) complexes, iodoaniline dyes, bis(S,O-dithiolene) complexes. Fluorescent proteins, such as green fluorescent protein (GFP) and modifications of GFP that have different absorption/emission properties are also useful. Complexes of certain rare earth metals (e.g., europium, samarium, terbium or dysprosium) are used in certain contexts, as are fluorescent nanocrystals (quantum dots).

Particular examples of chromophores which may be used include fluorescein, sulforhodamine 101 (Texas Red), rhodamine B, rhodamine 6G, rhodamine 19, indocyanine green, Cy2, Cy3B, Cy3.5, Cy5, Cy5.5, Cy7, Cy7.5, Marina Blue, Pacific Blue, Oregon Green 488, Oregon Green 514, tetramethylrhodamine, and Alexa Fluor 350, Alexa Fluor 430, Alexa Fluor 532, Alexa Fluor 546, Alexa Fluor 555, Alexa Fluor 568, Alexa Fluor 594, Alexa Fluor 633, Alexa Fluor 647, Alexa Fluor 660, Alexa Fluor 680, Alexa Fluor 700, and Alexa Fluor 750. Particularly preferred dyes are the cyanine dyes.

Particularly preferred are dyes which have absorption maxima in the visible or near infrared region, between 400 nm and 3 μm, particularly between 600 and 1300 nm.

Several relevant targets for endometriosis are enzymes. A contrast agent for optical imaging of endometriosis for targeting an enzyme can be an enzyme contrast agent substrate that can be transformed to a contrast agent product possessing different pharmacokinetic and/or pharmacodynamic properties from the contrast agent substrate. In this embodiment, the invention provides contrast agent substrates having affinity for an abnormally expressed enzyme, wherein the contrast agent substrate changes pharmacodynamic and/or pharmacokinetic properties upon chemical modification into a contrast agent product in a specific enzymatc transformation, and thereby enabling detection of areas of disease upon a deviation in the enzyme activity from the normal. Typical differences in pharmacodynamic and/or pharmacokinetic properties can be binding properties to specific tissues, membrane penetration properties, protein binding and solubility properties.

Alternatively, if the abnormally expressed target for diagnosis of endometriosis is an enzyme, the contrast agent for optical imaging can be a dye molecule that directly binds to the enzyme. The contrast agent will have affinity for the abnormally expressed enzyme, and this may be used to identify tissue or cells with increased enzymatic activity.

In a further aspect of the invention, the contrast agent changes dye characteristics as a result of an enzymatic transformation. For example, a fluorescent dye reporter of the contrast agent is quenched (no fluorescence) by associated quencher groups until an enzymatic cleavage takes place, separating the dye from the quencher groups and resulting in fluorescence at the site of the abnormally expressed enzyme.

Another aspect of this part of the invention is that the dye may change colour, as e.g. a change in absorption and/or emission spectrum, as a result of an enzymatic transformation.

If the abnormally expressed target for diagnosis of endometriosis is a receptor or another non-catalytical target, the contrast agent for optical imaging can bind directly to the target and normally not change the dye characteristics.

Another aspect of the invention is contrast agents for optical imaging of endometriosis characterized by having affinity for more than one abnormally expressed target related to the disease. Such contrast agents can have two or more different vectors or molecular subunits that target two or more different abnormally expressed targets.

The preferred contrast agents of the present invention are soluble in water. This means that the preferred contrast agents have a solubility in water at pH 7.4 of at least 1 mg/ml.

The contrast agents of the present invention can be identified by random screening, for example by testing of affinity for abnormally expressed targets of a library of dye labelled compounds either prepared and tested as single compounds or by preparation and testing of mixture of compounds (a combinatorial approach).

The contrast agents of the invention can also be identified by use of technology within the field of intelligent drug design. One way to perform this is to use computer-based techniques (molecular modelling or other forms of computer-aided drug design) or use of knowledge about natural and exogenous ligands (vectors)for the abnormally expressed targets. The sources for exogenous ligands can for example be the chemical structures of therapeutic molecules for targeting the same target. One typical approach here will be to bind the dye chemical sub-unit to the targeting vector so that the binding properties of the vector are not reduced. This can be performed by linking the dye at the far end, away from the pharmacophore centre (the active targeting part of the molecule). Alternatively, random screening may be used to identify suitable vectors before labelling with a reporter.

The contrast agents of the invention are preferably not endogenous substances alone. Some endogenous substances, for instance estrogen, have certain fluorescent properties in themselves, but they are not likely to be sufficient for use in optical imaging. Endogenous substances combined with an optical reporter however, falls within the contrast agents of the invention.

The contrast agents of the invention are intended for use in optical imaging. Any method that forms an image for diagnosis of disease, follow up of disease development or for follow up of disease treatment based on interaction with light in the electromagnetic spectrum from ultraviolet to near infrared radiation fall within the term optical imaging. Optical imaging further includes all methods from direct visualization without use of any device and use of devices such as various scopes, catheters and optical imaging equipment, for example computer based hardware for tomographic presentations. The contrast agents will be useful with optical imaging modalities and measurement techniques including, but not limited to: luminescence imaging; endoscopy; fluorescence endoscopy; optical coherence tomography; transmittance imaging; time resolved transmittance imaging; confocal imaging; nonlinear microscopy; photoacoustic imaging; acousto-optical imaging; spectroscopy; reflectance spectroscopy; interferometry; coherence interferometry; diffuse optical tomography and fluorescence mediated diffuse optical tomography (continuous wave, time domain and frequency domain systems), and measurement of light scattering, absorption, polarisation, luminescence, fluorescence lifetime, quantum yield, and quenching.

Examples of contrast agent for optical imaging of endometriosis according to the invention are shown below with some accompanied suggested synthetic routes:

Contrast Agents with Affinity for Angiogenesis:

Compound 1: 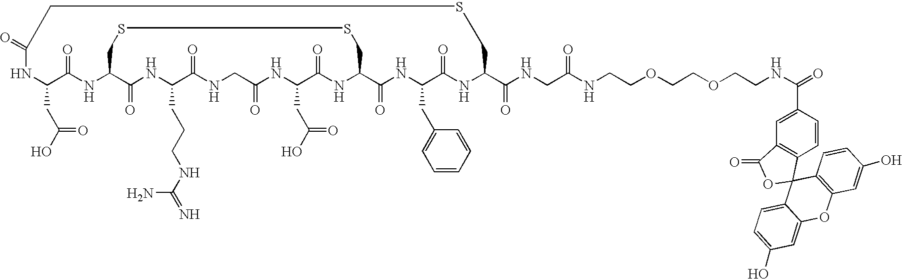
Vector: RGD-type peptide
Linker: PEG-moiety
Reporter: Fluorescein
   Compound II: 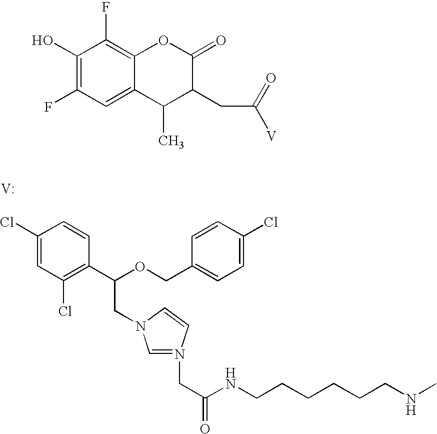
Wherein the reporter is a 7-hydroxycoumarine derivative.
   Contrast Agent with Affinity for Drogesterone Receptors:
   Compound III: 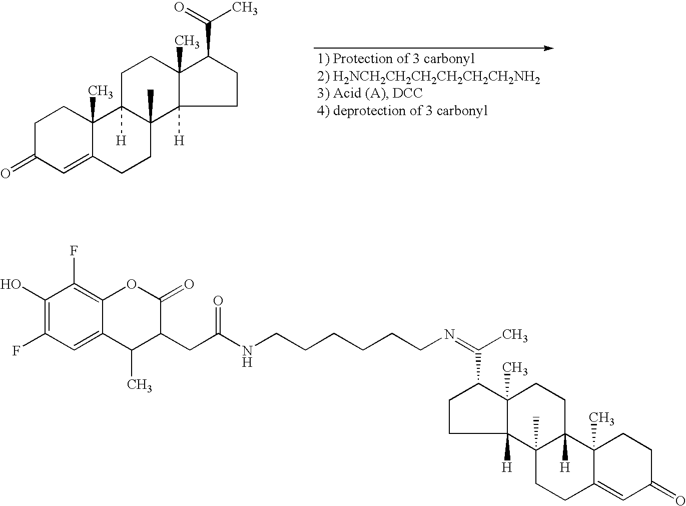
Wherein progesterone is coupled to a 7-hydroxycoumarine derivative (Acid (A)) via a alkyl-linker.
   Acid (A): 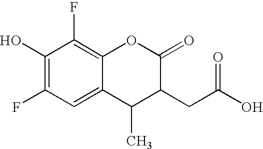
   Contrast Agent with Affinity for Estrogen Receptors:
   Compound IV:

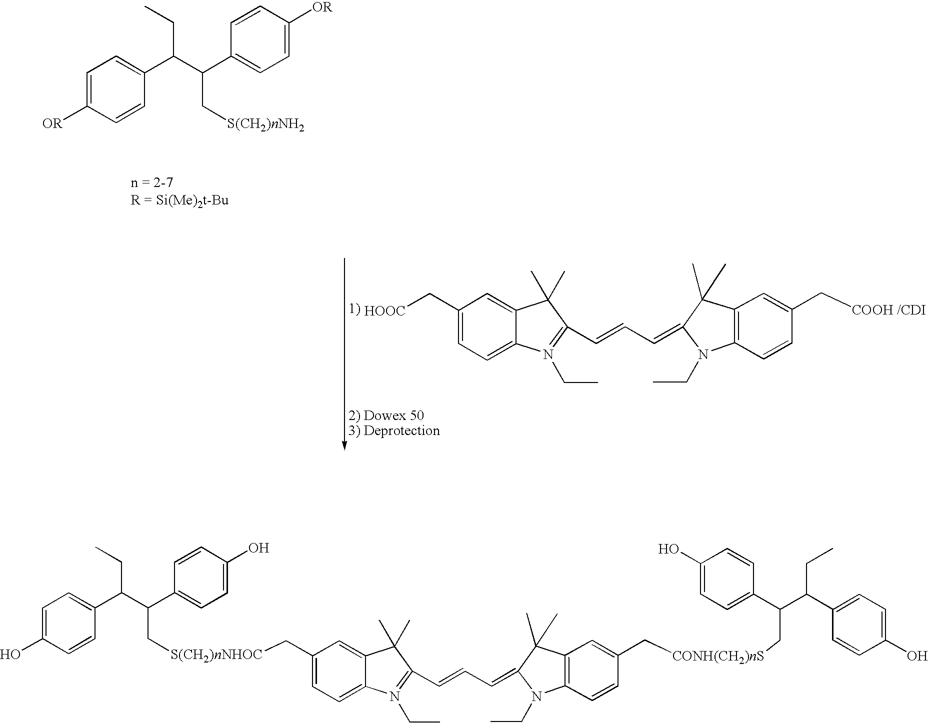

The compound has been described by Fevig et al in J. Med. Chem. 1987, 30, 156-165 but not in the context of optical imaging of endometriosis. The compound consists of two estrogen derivatives linked to a NIR dye.
Compound V:

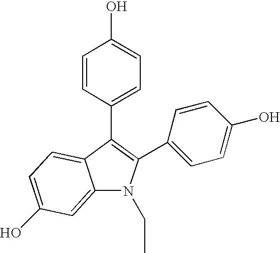

The compound has been described by Koulocheri et al in Eur. J. Org. Chem 2001, 1723-1729 but not in the context of optical imaging of endometriosis. The compound is a 2,3-bis(4-hydroxypenyl)indole having good estrogen binding properties and an intense long-wavelength fluorescent emission.

Contrast Agent with Affinity for Folate Binding Proteins:

Compound VI:

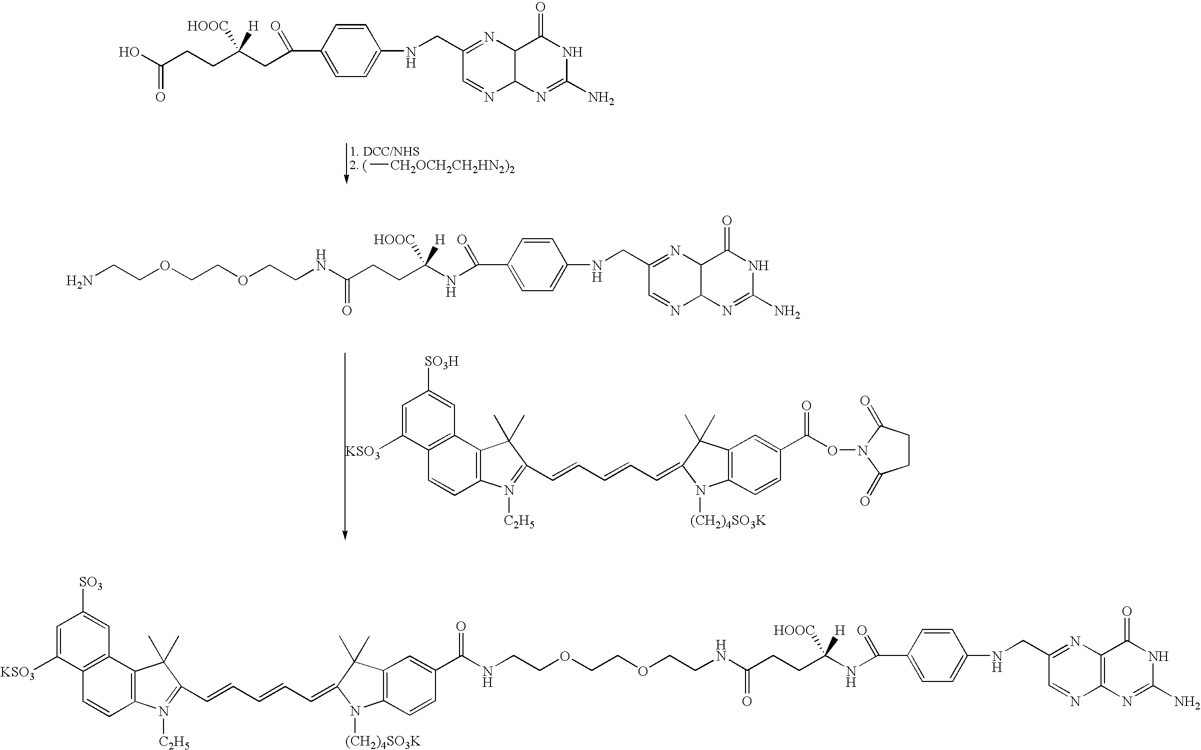

This complex comprising a NIR dye, a PEG moiety and a folate vector has been described in Bioconjugate Chem., Vol. 14, Ni.3, 2003, but not in the context of optical imaging of endometriosis.

A further embodiment is use of contrast agents of the invention for optical imaging of endometriosis, that is, for diagnosis of endometriosis, for follow up of the progress in endometriosis development or for follow up the treatment of endometriosis. In the context of this invention diagnosis includes screening of selected populations, early detection, biopsy guidance, characterisation, staging, grading, therapy efficacy monitoring, long-term follow-up of relapse and surgical guidance.

Still another embodiment of the invention is a method of optical imaging for diagnosis of endometriosis using the contrast agents as described.

Still another embodiment of the invention is a method of optical imaging for diagnosis, to follow up the progress of endometriosis development and to follow up the treatment of endometriosis.

One aspect of these methods is to administer the present contrast agents and follow the accumulation and elimination directly visually during surgery. Another aspect of these methods is to administer the present contrast agents and perform visual diagnosis through a laparoscope.

Still another aspect of the present invention is to administer the present contrast agents and perform the image diagnosis using computerized equipment as for example a tomograph.

Still another embodiment of the invention is use of a contrast agent as described for the manufacture of a diagnostic agent for use in a method of optical imaging of endometriosis involving administration of said diagnostic agent to an animate body and generation of an image of at least part of said body.

Still another embodiment of the invention is pharmaceutical compositions comprising one or more contrast agents as described or pharmaceutically acceptable salts thereof for optical imaging for diagnosis of endometriosis, for follow up progress of endometriosis development or for follow-up the treatment of endometriosis. The diagnostic agents of the present invention may be formulated in conventional pharmaceutical or veterinary parenteral administration forms, e.g. suspensions, dispersions, etc., for example in an aqueous vehicle such as water for injections. Such compositions may further contain pharmaceutically acceptable diluents and excipients and formulation aids, for example stabilizers, antioxidants, osmolality adjusting agents, buffers, pH adjusting agents, etc. The most preferred formulation is a sterile solution for intravascular administration or for direct injection into area of interest. Where the agent is formulated in a ready-to-use form for parenteral administration, the carrier medium is preferably isotonic or somewhat hypertonic.

The dosage of the optical diagnostic agents of the invention will depend upon the clinical indication, choice of contrast agent and method of administration. In general, however dosages will be between 10 μg and 5 grams for an adult human.

While the present invention is particularly suitable for methods involving parenteral administration of the contrast agent, e.g. into the vasculature or directly into an organ of muscle tissue, intravenous administration being especially preferred, it is also applicable where administration is not via a parenteral route, e.g. where administration is transdermal, nasal, sub-lingual or is into an externally voiding body cavity, e.g. the gi tract, the bladder, the uterus or the vagina. The present invention is deemed to extend to cover such administration.

The following examples are illustrative only and not intended to be limiting. Other features and advantages of the invention will be apparent from the detailed description and from the claims.

### EXAMPLES

### Example 1

### Contrast Agent with Affinity for Folate Binding Protein. Synthesis of Folic Acid—Fluorescin Linker Conjugate

Step 1

Folio acid dehydrate (1.0 g) is mixed with toluene (500 ml) and heated to 110° C. The mixture is cooled to 50° C. and evaporated to dryness. Folio acid anhydrate is isolated.

Step 2

Folio acid anhydrate (441 mg, 1 mmol) and 1,3-dicyclohexylcarbodiimide (DCC) (226 mg, 1.1 mmol) are dissolved in DMF (30 ml). The mixture is cooled to 0° C. and a solution of 2,2′-ethylenedioxy)bis(ethylamine) (296 mg, 2 mmol) and DMAP (30 mg) in DMF(10 ml) is added. The mixture is stirred for 1 hour at 0° C. and then stirred for 72 hours at ambient temperature. The solution is evaporated and the conjugate between folio acid and the bisamide is isolated as monoamide by chromatography (silica, chloroform and methanol).

Step 3

5(6) - Carboxyfluorescein (188 mg, 0.5 mmol) and dicyclohexylcarbodiimide (113 mg, 0.55 mmol) are dissolved in DMF (20 ml). The mixture is cooled to 0° C. and a solution of the monoamide from step 2 above (286 mg, 0.5 mmol) and DMAP (15 mg) in DMF (5 ml) is added. The mixture is stirred for 1 hour at 0° C. and then stirred for 72 hours at ambient temperature. The solution is evaporated and the final conjugate is isolated by chromatography (silica, chloroform and methanol).

### Example 2

### Contrast Agent for Mapping of Matrix Metalloproteinase (MMP). Synthesis of Fluorescein-Cys-Gly-Pro-Leu-Gly-Leu-Leu-Ala-Arg-OH Linker Conjuaate

Step 1

The peptide component was synthesised on an ABI 433A automatic peptide synthesiser starting with Fmoc -Arg (Pmc) -wang resin on a 0.1 mmol scale using 1 mmol amino acid cartridges. The amino acids were pre-activated using HBTU before coupling. An aliquot of the peptide resin was then transferred to a clean round bottom flask an N-methyl morpholine (1 mmol) in DMF (5 ml) added followed by chloroacetyl chloride (1 mmol). The mixture was gently shaken until Kaiser test negative. The resin was extensively washed with DMF.

Step 2

5(6)-carboxyfluorescein (188 mg, 0.5.mmol) and dicyclohexylcarbodiimide (113 mg, 0.55 mmol) are dissolved in DMF (20 ml). The mixture is stirred for 2 hours and cooled to 0° C. A solution of hexamethylenediamide (116 mg, 1 mmol) and DMAP (30 mg) in DMF is added and the mixture is stirred at ambient temperature for 72 hours. The solution is evaporated and the conjugate between carboxyfluorescein and hexamethylene-amine is isolated as monoamide by chromatography (silica, chloroform and methanol).

Step 3

The resin from step 1 is suspended in DMF (5 ml) and amide-amine conjugate from step 2 (0.5 mmol)pre-dissolved in DMF (5 ml) containing triethylamine (0.5 mmol) is added. The mixture is heated to 50° C. for 16 hours then excess reagents filtered off, following extensive washing with DMF, DCM and diethyl ether then air drying. The product is treated with TFA containing TIS (5%), H₂O (5%), and phenol (2.5%) for 2 hours.

Excess TFA is removed in vacuo and the peptide is precipitated by the addition of diethyl ether. The crude peptide conjugate is purified by preparative HPLC C C-18, acetonitril, TFA, water).

### Example 3

### Contrast Agent for Mapping of Estrogen Receptors; Cy 5.5 Linked to Estrogen Derivative

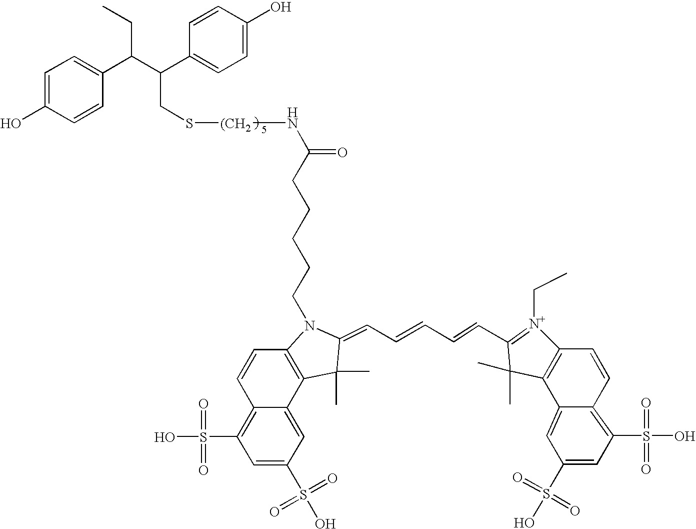

(2R, 3S)-2,3-bis[4-[(tert-butyldimethylsilyl)oxy]-phenyl]pentyl-5-aminopenylsulfide is prepared according to T. L. Fevig et al in J.Med.Chem 1987, 30, 156-165.

The above amine (1 mmol) and Cy 5.5 NHS ester (1 mmol) are dissolved in dimethylformamide (25) ml). N-methylmorpholine (300 μl) is added and the mixture stirred at ambient temperature for 4 days. The mixture is evaporated and the coupling product is isolated by flash chromoatography (silica, hexane and ethyl acetate). This product is dissolved in ethylacetate and treated with paratoluenesulphonic acid (25 mmol). The solvent is evaporated and the oily mixture is heated at 40° C. for 30 minutes. The product is isolated by flash chromatography (silica, hexane and ethyl acetate).

### Example 4

### Contrast Agent with Affinity for Integrins: RGD Peptide Linked to Cy5.5

Step 1. Assembly of Amino Acids

The peptide sequence Asp-D-Phe-Lys-Arg-Gly was assembled on an Applied Biosystems 433A peptide synthesizer starting with 0.25 mmol Fmoc-Gly-SASRIN resin. An excess of 1 mmol pre-activated amino acids (using HBTU; O-Benzotriazol-1-yl-N,N,N′,N′-tetramethyluronium hexafluorophosohate) was applied in the coupling steps. The cleavage of the fully protected peptide from the resins was carried out by treatment of the resin with three portions of 35 mL of 1% trifluoroacetic acid (TFA) in dichloromethane (DCM) for 5 minutes each. The filtrates containing the peptide was immediately neutralised with 2% piperidine in DCM. The organics were extracted with water (3×100 mL), dried with MgSO₄ and evaporated in vacuo. Diethyl ether was added to the residue and the precipitate washed with ether and air-dried affording 30 mg of crude protected peptide. The product was analysed by analytical HPLC (conditions: Gradient, 20-70% B over 10 min where A=H₂O/0.1% TFA and B=CH₃CN/0.1% TFA; flow, 2 mL/min; column, Phenomenex Luna 3μ 5×4.6 mm; detection, UV 214 nm; product retention time 7.58 min). Further product characterisation was carried out using electrospray mass spectrometry (MH⁺ calculated, 1044.5; MH⁺ found, 1044.4).
Step 2. N-C Cyclisation

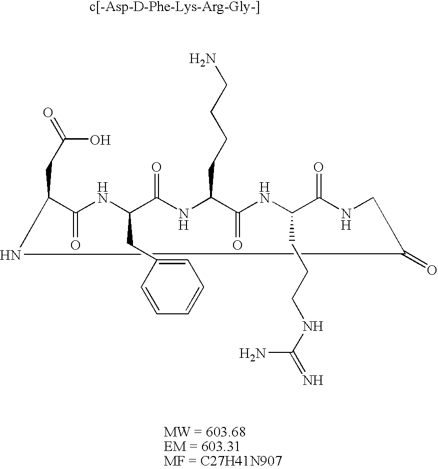

30 mg of the fully protected peptide, 16 mg of PyAOP, 4 mg of HOAt and 6 μL of N-methylmorpholine (NMM) were dissolved in dimethylformamide/DCM (1:1) and stirred over night. The mixture was evaporated in vacuo and diethyl ether added to the residue. The precipitate was washed with ether and air-dried. The crude cyclic fully protected peptide was treated with a solution of 25 mL TFA containing 5% water, 5% triisopropylsilane and 2.5% phenol for two hours. TFA was evaporated in vacuo and diethyl ether added to the residue. The precipitate was washed with ether and air-dried. Purification by preparative RP-HPLC (0-30% B over 40 min, where A H₂O/0.1% TFA and B=CH₃CN/0.1% TFA, at a flow rate of 10 mL/min on a Phenomenex Luna 5μ C18 250×21.20 mm column) of the crude material afforded 2.3 mg pure product peptide. The pure product was analysed by analytical HPLC (conditions: Gradient, 0-15% B over 10 min where A=H-₂O/0.1% TFA and B=CH₃CN/0.1% TFA; flow, 2 ml /min; column, Phenomenex Luna 3μ 5×4.6 mm; detection, UV 214 nm; product retention time 6.97 min). Further product characterisation was carried out using electrospray mass spectrometry (MH⁺ calculated, 604.3; MH⁺ found, 604.4).
Step 3. Conjugation of Cy5.5 to RGD Peptide

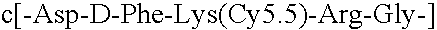

0.6 mg of the RGD peptide, 1.7 mg of Cy5.5 mono NHS ester and 5 μL of NMM were dissolved in 1 mL of dimethylformamide (DMF) and the reaction mixture stirred for 2 hrs. Diethyl ether was added to the DMF solution and the blue precipitate washed with diethyl ether and air-dried affording 0.7 mg of crude RGD peptide conjugated to Cy5.5.The pure product was analysed by analytical HPLC (conditions: Gradient, 5-50% B over 10 min where A=H₂O/0.1% TFA and B=CH₃CN/0.1% TFA; flow, 0.3 mL/min; column, Phenomenex Luna 3μ 5×2 mm; detection, UV 214 nm; product retention time 8.32 min). Further product characterisation was carried out using electrospray mass spectrometry (MH⁺ calculated, 1502.5; MH⁺ found, 1502.6).

What is claimed is:

1-12. (canceled)

13. An optical imaging contrast agent with affinity for an abnormally expressed biological target associated with endometrioses, of formula I
V-L-R (I)
wherein V is one or more vector moieties having affinity for an abnormally expressed target in endometriosis, L is a linker moiety or a bond and R is one or more reporter moieties detectable in in vivo optical imaging, and wherein the contrast agent has a molecular weight below 10000 Daltons.

14. A contrast agent as claimed in claim 13 comprising a contrast agent substrate, wherein the target is an abnormally expressed enzyme, such that the contrast agent changes pharmacodynamic properties and/or pharmacokinetic properties upon a chemical modification from a contrast agent substrate to a contrast agent product upon a specific enzymatic transformation.

15. A contrast agent as claimed in claim 14 wherein the contrast agent changes binding properties to specific tissue, membrane penetration properties, protein binding or solubility properties upon the chemical modification.

16. A contrast agent as claimed in claim 13 having affinity for any of the targets selected from the group of angiogenesis targets, adhesion molecules, estrogen receptors, progesterone receptors, Cathepsin H and Cathepsin S, aromatase, reductase, CD10, endoglin, haptoglobin and cyclin D2.

17. A contrast agent as claimed in claim 13 wherein V is selected from peptides, peptoid moieties, oligonucleotides, oligosaccharides and lipid-related compounds and traditional organic drug-like small molecules.

18. A contrast agent as claimed in claim13 wherein R is a dye that interacts with light in the wavelength region from the ultraviolet to the infrared part of the electromagnetic spectrum.

19. A contrast agent as claimed in claim 13 wherein R is a cyanine dye.

20. A pharmaceutical composition for optical imaging for diagnosis of endometriosis, for follow up of progress of endometriosis, or for follow up of treatment of endometriosis, comprising a contrast agent as defined in claim 13 together with at least one pharmaceutically acceptable carrier or excipient.

21. A method of generating an optical image of an animate subject involving administering a contrast agent to said subject and generating an optical image of at least a part of said subject to which said contrast agent has distributed, characterized in a contrast agent as defined in claim 13 is used.

22. A method of generating an optical image of an animate subject involving administering a contrast agent to said subject and generating an optical image of at least a part of said subject to which said contrast agent has distributed for a diagnosis of endometriosis, wherein the follow up of the progress of endometriosis development or the follow up treatment of endometriosis is obtained by using a contrast agent as defined in claim 13.


SEQUENCE LISTING (5 sequences)

SEQ ID NO: 1 - protein, 9 aa, Artificial sequence
  Synthesised peptide
CGPLGLLAR

SEQ ID NO: 2 - protein, 5 aa, Artificial sequence
  Syntehsised peptide
DFKRG

SEQ ID NO: 3 - protein, 5 aa, Artificial sequence
  Synthesised peptide
  MISC_FEATURE (1)..(5): Cyclisation of peptide through peptide bond between amino acid 1 and 5
DFKRG

SEQ ID NO: 4 - protein, 8 aa, Artificial sequence
  Synthesised peptide
DCRGDCFC

SEQ ID NO: 5 - protein, 8 aa, Artificial sequence
  Synthesised peptide
  THIOETH (1)..(8): Thioether bridge between amino acids 1 and 8
  DISULFID (2)..(6): Disulphide bonf between amino acids 2 and 6
DCRGDCFC